(19) United States
(12) Patent Application Publication
(10) Pub. No.: US 2019/0150894 A1
(43) Pub. Date: May 23, 2019

(54) CONTROL DEVICE, CONTROL METHOD, CONTROL SYSTEM, AND NON-TRANSITORY STORAGE MEDIUM

(71) Applicant: CANON KABUSHIKI KAISHA, Tokyo (JP)

(72) Inventors: Kensuke Kato, Tokyo (JP); Nobu Miyazawa, Yokohama-shi (JP); Hiroshi Arai, Tokyo (JP)

(21) Appl. No.: 16/239,330

(22) Filed: Jan. 3, 2019

Related U.S. Application Data
(63) Continuation of application No. PCT/JP2017/024575, filed on Jul. 5, 2017.

(30) Foreign Application Priority Data
Jul. 8, 2016 (JP) 2016-136107
Nov. 25, 2016 (JP) 2016-229311

(51) Int. Cl. A61B 8/00; A61B 5/00; A61B 8/14

(52) U.S. Cl. CPC A61B 8/4416; A61B 5/0095; A61B 8/54; A61B 8/4254; A61B 8/463; A61B 8/14

(57) ABSTRACT

A control device outputs an ultrasonic signal by transmission and reception of an ultrasonic wave relative to an object, obtains an ultrasonic signal and a photoacoustic signal from a probe which outputs the photoacoustic signal by receiving a photoacoustic wave generated due to light irradiation onto the object, obtains information on displacement of the probe, and displays a photoacoustic image on a display unit based on the information on displacement of the probe.

### CROSS-REFERENCE TO RELATED APPLICATIONS

This application is a Continuation of International Patent Application No. PCT/JP2017/024575 filed Jul. 5, 2017, which claims the benefit of Japanese Patent Application No. 2016-136107 filed Jul. 8, 2016 and No. 2016-229311 filed Nov. 25, 2016, all of which are hereby incorporated by reference herein in their entirety.

### TECHNICAL FIELD

The present invention relates to a control device, a control method, a control system, and a non-transitory storage medium.

### BACKGROUND ART

As an imaging apparatus which images a state of an inside of an object in a minimally invasive manner, an ultrasonic imaging apparatus or a photoacoustic imaging apparatus has been used. PTL 1 discloses a photoacoustic measurement apparatus capable of performing switching between an operation mode including detection of a photoacoustic signal and an operation mode which does not include the detection of a photoacoustic signal by means of an operation performed on a mode switch included in a probe.

### CITATION LIST

### Patent Literature

PTL 1 Japanese Patent Laid-Open No. 2012-196430

In an imaging apparatus which obtains an ultrasonic signal and a photoacoustic signal, it is assumed that imaging is performed while switching of an operation mode associated with detection of an ultrasonic signal or a photoacoustic signal is performed. However, in a case where a mode switch included in a probe is to be operated so that switching of an operation mode is performed, a user may interrupt an operation being performed on the probe. The user may not observe a desired image if the object moves or a position of the probe is shifted during the interruption.

### SUMMARY OF INVENTION

The present invention provides a control device including first obtaining means for outputting an ultrasonic signal by transmission and reception of an ultrasonic wave relative to an object and obtaining the ultrasonic signal and a photoacoustic signal using a probe which outputs the photoacoustic signal by receiving a photoacoustic wave generated due to light irradiation onto the object, second obtaining means for obtaining information on displacement of the probe, and display control means for displaying a photoacoustic image generated using the photoacoustic signal on a display unit based on the information on displacement.

Further features of the present invention will become apparent from the following description of exemplary embodiments with reference to the attached drawings.

### BRIEF DESCRIPTION OF DRAWINGS

FIG. 1 is a diagram illustrating an example of a configuration of a system including a control device according to an embodiment of the present invention.

FIG. 2 is a diagram illustrating an example of a hardware configuration of the control device according to the embodiment of the present invention.

FIG. 3 is a diagram illustrating an example of a functional configuration of the control device according to the embodiment of the present invention.

FIGS. 4A to 4C are diagrams illustrating examples of images displayed on a display unit by the control device according to the embodiment of the present invention.

FIG. 5 is a diagram illustrating an example of a configuration including a control device according to a first embodiment.

FIG. 6 is a flowchart of an example of a process performed by the control device according to the first embodiment.

FIG. 7 is a diagram illustrating an example of a configuration including the control device according to the first embodiment.

FIG. 8 is a diagram illustrating an example of a configuration including a control device according to a second embodiment.

FIG. 9 is a flowchart of an example of a process performed by the control device according to the second embodiment.

FIG. 10 is a flowchart of an example of a process performed by a control device according to a third embodiment.

FIG. 11 is a flowchart of an example of a process performed by a control device according to an embodiment of the present invention.

FIGS. 12A and 12B are flowcharts of examples of a process performed by a control device according to an embodiment of the present invention.

### DESCRIPTION OF EMBODIMENTS

Hereinafter, embodiments of the present invention will be described hereinafter with reference to the accompanying drawings.

### First Embodiment

In this specification, an acoustic wave which is generated due to expansion caused in an object by irradiation with light on the object is referred to as a photoacoustic wave. Furthermore, an acoustic wave transmitted from a transducer or a reflection wave (echo) obtained when the transmitted acoustic wave is reflected in an inside of the object is referred to as an ultrasonic wave.

As a method for imaging a state of an inside of an object in a minimally invasive manner, a method for imaging using ultrasonic waves and a method for imaging using photoacoustic waves have been used. As the method for imaging using ultrasonic waves, an image is generated based on a period of time in which an ultrasonic wave oscillated by a transducer is reflected in accordance with a difference between acoustic impedances in a tissue in the object and a resultant reflection wave reaches the transducer and intensity of the reflection wave, for example. An image generated using ultrasonic waves is referred to as an ultrasonic image hereinafter. A user operates a probe while changing an angle of the probe or the like so as to observe ultrasonic images of various cross sections in real time. A shape of an internal organ or a tissue is rendered in an ultrasonic image which is utilized for finding of a tumor. Furthermore, as the method for imaging using photoacoustic waves, an image is generated based on ultrasonic waves (photoacoustic waves) generated due to adiabatically-expansion tissue in the object which is irradiated with light. An image generated using photoacoustic waves is referred to as a photoacoustic image hereinafter. Information associated with an optical characteristic, such as degrees of light absorption in tissues is rendered in a photoacoustic image. For example, a blood vessel may be rendered in a photoacoustic image due to an optical characteristic of hemoglobin, and utilization for evaluation of a malignancy degree of a tumor has been discussed.

To enhance accuracy of diagnosis, various information may be collected so that different phenomena in the same portion of the object are imaged based on different principles. For example, form information obtained by a computed tomography (CT) image and function information associated with metabolism obtained in a positron emission tomography (PET) image may be combined with each other for diagnosis of a cancer. In this way, the diagnosis using information obtained by generating images of different phenomena based on different principles is seen to be effective for improvement of accuracy of diagnosis.

An imaging apparatus which obtains an image by combining features of the ultrasonic image and the photoacoustic image described above has been discussed. In particular, both of the ultrasonic image and the photoacoustic image are generated using ultrasonic waves from the object, and therefore, imaging of the ultrasonic image and imaging of the photoacoustic image may be performed by the same imaging apparatus. More specifically, a reflection wave obtained by irradiating the object with light and a photoacoustic wave may be received by the same transducer. Therefore, an imaging apparatus which is capable of obtaining an ultrasonic signal and a photoacoustic signal by a single probe and which performs imaging of an ultrasonic image and imaging of a photoacoustic image may be realized without a complicated hardware configuration.

It is assumed that the user desires to operate a probe similarly to general imaging of an ultrasonic image in such an imaging apparatus which performs imaging of an ultrasonic image and imaging of a photoacoustic image. Specifically, the user may bring the probe into contact with a surface of the object and operate the probe while observing an image displayed based on information obtained by the probe. In this case, if switching of an operation mode associated with a signal obtainment and image display is performed using a switch disposed on the probe or an input device disposed on a console of the imaging apparatus, the user is required to interrupt the probe operation performed while observing an image. Therefore, a movement of an object may occur between input operations performed on the input device using the switch or the console or a position of the probe may be shifted.

For example, a case where a malignancy degree of a tumor is evaluated by observing the ultrasonic image and the photoacoustic image as a pair is considered. It is assumed that, when the user operates a probe while observing an ultrasonic image, a portion which may be a tumor is found, and therefore, the user desires to collect information on a blood vessel by obtaining a photoacoustic image. In this case, the probe may be shifted from a position in which a portion of a possible tumor may be observed while an operation input is performed on an input device, such as a switch or a console, described above so that switching to an operation mode for displaying a photoacoustic image is performed. An object of a first embodiment is to provide a control device capable of switching an image to be displayed without degrading operability at a time when the user observes an image.

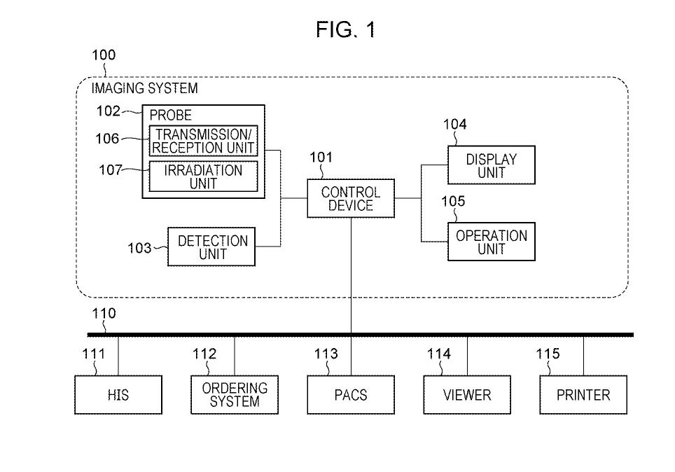

FIG. 1 is a diagram illustrating an example of a configuration of a system including a control device 101 according to the first embodiment. An imaging system 100 capable of generating an ultrasonic image and a photoacoustic image is connected to various external apparatuses through a network 110. Various components included in the imaging system 100 and the various external apparatuses may not be installed in the same facility and are at least connected to one another in a communication available manner.

The imaging system 100 includes the control device 101, a probe 102, a detection unit 103, a display unit 104, and an operation unit 105. The control device 101 obtains an ultrasonic signal and a photoacoustic signal and is capable of displaying an ultrasonic image and a photoacoustic image on the display unit 104 based on information associated with a movement of the probe 102 obtained by the detection unit 103. Furthermore, the control device 101 obtains information associated with an examination including imaging of the ultrasonic image and the photoacoustic image from an ordering system 112 and controls the probe 102, the detection unit 103, and the display unit 104 when the examination is performed. The control device 101 outputs the generated ultrasonic image, the generated photoacoustic image, and a superimposed image obtained by superimposing the photoacoustic image on the ultrasonic image to a PACS 113. The control device 101 performs transmission and reception of information relative to an external apparatus, such as the ordering system 112 or the PACS 113, based on a standard of health level 7 (HL7) or digital imaging and communications in medicine (DICOM). A process performed by the control device 101 will be described in detail hereinafter.

The probe 102 is operated by the user and transmits the ultrasonic signal and the photoacoustic signal to the control device 101. The probe 102 includes a transmission/reception unit 106 and an irradiation unit 107. The probe 102 transmits an ultrasonic wave from the transmission/reception unit 106 and receives a reflection wave by the transmission/reception unit 106. Furthermore, the probe 102 irradiates the object with light from the irradiation unit 107 and receives a photoacoustic wave by the transmission/reception unit 106. The probe 102 converts the received reflection wave and the photoacoustic wave into electric signals, that is, an ultrasonic signal and a photoacoustic signal, to be transmitted to the control device 101. The probe 102 is preferably controlled such that transmission of an ultrasonic wave is performed to obtain an ultrasonic signal and light irradiation is performed to obtain a photoacoustic signal, when information indicating contact to the object is received. The probe 102 may obtain an ultrasonic signal and a photoacoustic signal alternately or simultaneously or may obtain an ultrasonic signal and a photoacoustic signal in a predetermined manner.

The transmission/reception unit 106 includes at least one transducer (not illustrated), a matching layer (not illustrated), a damper (not illustrated), and an acoustic lens (not illustrated). The transducer (not illustrated) is formed of a substance having a piezoelectric effect, such as a lead zirconate titanate (PZT) or polyvinylidene difluoride (PVDF). The transducer (not illustrated) may not be of a piezoelectric element and may be a capacitive micro-machined ultrasonic transducer (CMUT) or a transducer using a Fabry-Perot interferometer. Typically, the ultrasonic signal includes frequency components in a range from 2 to 20 MHz and the photoacoustic signal includes frequency components in a range from 0.1 to 100 MHz, and therefore, a transducer (not illustrated) capable of detecting these frequencies is used. The signal obtained by the transducer (not illustrated) is a time-resolved signal. Amplitudes of the received signals indicate values based on sound pressures received by the transducer at various time points. The transmission/reception unit 106 includes a circuit (not illustrated) for an electronic focus or a controller. Transducers (not illustrated) are arranged in a sector, a linear array, convex, an annular array, or a matrix array.

The transmission/reception unit 106 may include an amplifier (not illustrated) which amplifies a time-series analog signal received by the transducer (not illustrated). Furthermore, the transmission/reception unit 106 may include an A/D converter which converts the time-series analog signal received by the transducer (not illustrated) into a time-series digital signal. The transducers (not illustrated) may be divided into for transmission and for reception depending on a purpose of imaging of an ultrasonic wave image. Alternatively, the transducers (not illustrated) may be divided into for imaging of an ultrasonic wave and for imaging of a photoacoustic image.

The irradiation unit 107 includes a light source (not illustrated) for obtaining a photoacoustic signal and an optical system (not illustrated) for guiding pulse light emitted from the light source (not illustrated) to the object. The light emitted from the light source (not illustrated) has a pulse width is 1 ns or more and 100 ns or less. Furthermore, the light emitted from the light source (not illustrated) has a wavelength of 400 nm or more and 1600 nm or less. When a blood vessel positioned in the vicinity of a surface of the test body is to be imaged in high resolution, a wavelength is preferably in a range from 400 nm inclusive to 700 nm inclusive which has is considerably absorbed in the blood vessel. Furthermore, when a depth portion of the object is to be imaged, a wavelength is preferably in a range from 700 nm inclusive to 1100 nm inclusive which is difficult to be absorbed in a tissue, such as water or fat.

The light source (not illustrated) is laser or a light emitting diode, for example. The irradiation unit 107 may include a light source in which a wavelength may be changed so as to obtain a photoacoustic signal using light of a plurality of wavelengths. Alternatively, the irradiation unit 107 may include a plurality of light sources which generate different light beams of different wavelengths and alternately emit the light beams of the different wavelengths from the light sources. The laser is solid state laser, gas laser, dye laser, or semiconductor laser, for example. As the light source (not illustrated), pulse laser, such as Nd:YAG laser or alexandrite laser, may be used. Furthermore, Ti:sa laser or optical parametric oscillator (OPO) laser which sets light of Nd:YAG laser as excitation light may be used as the light source (not illustrated). Furthermore, a microwave source may be used as the light source (not illustrated).

As the optical system (not illustrated), an optical element, such as a lens, a mirror, or an optical fiber, is used. In a case where the object is a breast, a beam diameter of pulse light is preferably enlarged in irradiation, and therefore, the optical system (not illustrated) may have a diffuser panel which diffuses light. The optical system (not illustrated) may include a lens or the like so as to focus a beam to improve resolution.

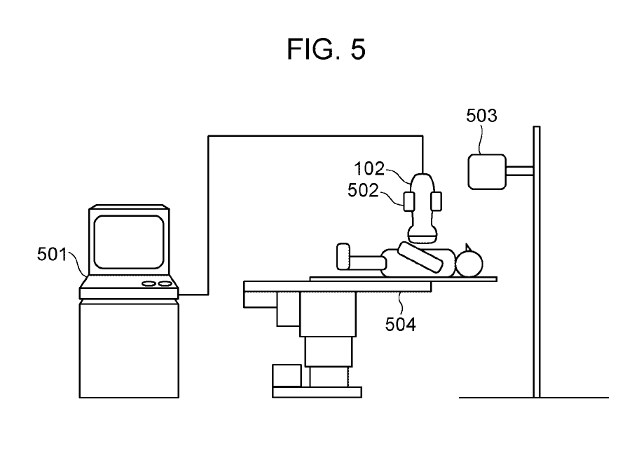

The detection unit 103 obtains information on displacement of the probe 102. According to the first embodiment, a case where the detection unit 103 includes a magnetic transmitter 503 and a magnetic sensor 502 which are illustrated in FIG. 5 will be described as an example. The detection unit 103 obtains, as information on a movement of the probe 102, information on a speed of a movement of the probe 102 relative to the object, information on a rotation speed of the probe 102, and information on a degree of a pressure applied to the object. The detection unit 103 transmits the obtained information on a movement of the probe 102 to the control device 101.

The display unit 104 displays an image captured by the imaging system 100 and information on an examination under control of the control device 101. The display unit 104 provides an interface which receives an instruction issued by the user under control of the control device 101. The display unit 104 is a liquid crystal display, for example.

The operation unit 105 transmits information on an input of a user operation to the control device 101. The operation unit 105 includes a keyboard, a trackball, or various buttons for performing operation inputs associated with the examination.

Note that the display unit 104 and the operation unit 105 may be integrated as a touch panel display. Furthermore, the control device 101, the display unit 104, and the operation unit 105 may not be separately provided and may be integrated as illustrated as a console 501 of FIG. 5. The control device 101 may include a plurality of probes.

A hospital information system (HIS) 111 assists services of a hospital. The HIS 111 includes an electronic health record system, an ordering system, and a medical accounting system. The HIS 111 may manage a series of operations from order issuance of an examination to accounting. The ordering system of the HIS 111 transmits order information to the ordering system 112 for each department. The ordering system 112 described below manages execution of the order.

The ordering system 112 manages examination information and manages progresses of examinations in imaging apparatuses. The ordering system 112 may be configured for each department which performs an examination. The ordering system 112 is a radiology information system (RIS) in a radiation department, for example. The ordering system 112 transmits information on an examination to be performed by the imaging system 100 to the control device 101 in response to an inquiry supplied from the control device 101. The ordering system 112 receives information on a progress of the examination from the control device 101. When receiving information indicating completion of the examination from the control device 101, the ordering system 112 transmits the information indicating completion of the examination to the HIS 111. The ordering system 112 may be integrated with the HIS 111.

A picture archiving and communication system (PACS) 113 is a database system which stores images obtained by the various imaging apparatuses installed out of the facility. The PACS 113 includes a storage unit (not illustrated) which stores a medical image, an imaging condition for the medical image, supplemental information including parameters for image processing including reconfiguration and patient information and includes a controller (not illustrated) which manages the information stored in the storage unit. The PACS 113 stores an ultrasonic image, a photoacoustic image, and a superimposed image output from the control device 101. Communication between the PACS 113 and the control device 101 and various images stored in the PACS 113 may be preferably based on a standard, such as HL7 or DICOM. The various images output from the control device 101 have various tags having supplemental information based on the DICOM standard.

A viewer 114 is a terminal for image diagnosis which reads an image stored in the PACS 113 or the like and displays the image for diagnosis. A doctor observes the image displayed in the viewer 114 and records information obtained as a result of the observation in an image diagnosis report. The image diagnosis report generated by the viewer 114 may be stored in the viewer 114 or may be output to the PACS 113 or a report server (not illustrated) which stores the image diagnosis report.

A printer 115 prints an image stored in the PACS 113 or the like. The printer 115 is a film printer, for example, which outputs an image stored in the PACS 113 or the like by printing the image on a film.

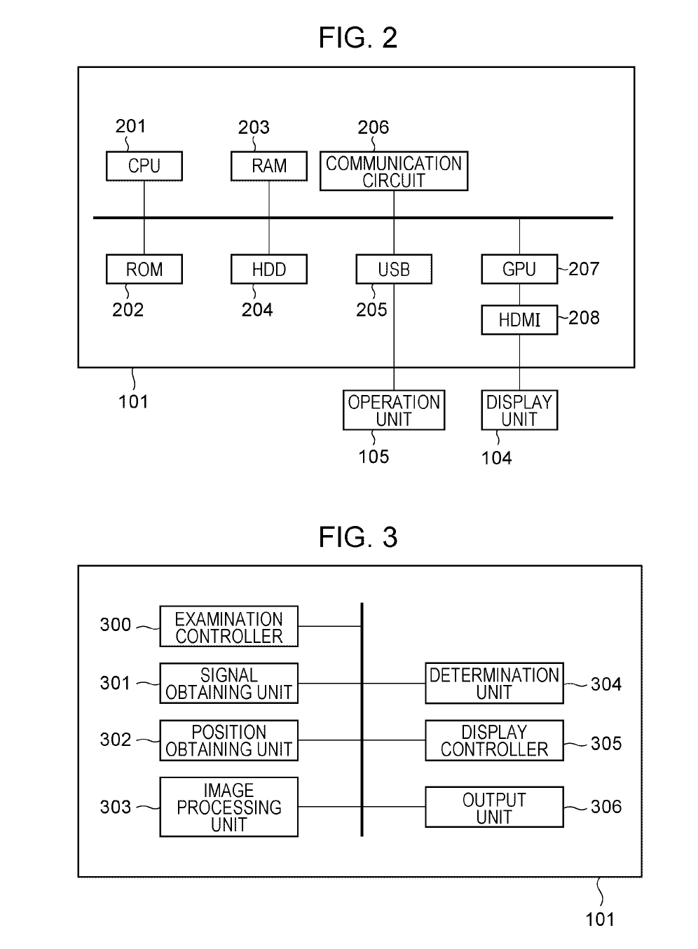

FIG. 2 is a diagram illustrating an example of a hardware configuration of the control device 101. The control device 101 includes a CPU 201, a ROM 202, a RAM 203, an HDD 204, a USB 205, a communication circuit 206, a GPU board 207, and an HDMI (registered trademark) 208. The units are connected to one another by an internal bus in a communication available manner.

The CPU (central processing unit) 201 is a control circuit which integrally controls the control device 101 and the units connected to the control device 101. The CPU 201 performs the control by executing programs stored in the ROM 202. Furthermore, the CPU 201 executes a display driver which is software for controlling the display unit 104 so as to perform display control on the display unit 104. Furthermore, the CPU 201 performs input/output control relative to the operation unit 105.

The ROM (read only memory) 202 stores programs and data which store a procedure of the control performed by the CPU 201.

The RAM (random access memory) 203 stores programs for executing a process of the control device 101 and processes of the units connected to the control device 101 and various parameters used in image processing. The RAM 203 stores control programs to be executed by the CPU 201 and temporarily stores various data to be used when the control device 101 executes various control operations.

The HDD (hard disk drive) 204 is an auxiliary storage device which stores various data including an ultrasonic wave image and a photoacoustic image.

The USB (universal serial bus) 205 is a connection unit connected to the operation unit 105.

The communication circuit 206 is used to communicate with the units included in the imaging system 100 and various external apparatuses connected to the network 110. The communication circuit 206 may be realized by a plurality of configurations depending on a desired communication form.

The GPU board 207 is a general graphics board including a GPU and a video memory. The GPU board 207 constitutes a portion of the image processing unit 303 or the entire image processing unit 303 and performs a reconfiguration process on a photoacoustic image, for example. Use of such a calculation device, calculation of the reconfiguration process and the like may be performed at high speed without using dedicated hardware.

A high definition multimedia interface (HDMI) (registered trademark) 208 is a connection unit connected to the display unit 104.

The CPU 201 and the GPU are examples of a processor. Furthermore, the ROM 202, the RAM 203, and the HDD 204 are examples of a memory. The control device 101 may have a plurality of processors. In the first embodiment, when the processor included in the control device 101 executes programs stored in the memory, functions of the units included in the control device 101 are realized.

Note that the control device 101 may include a CPU or a GPU which performs a specific process in a dedicated manner. Furthermore, the control device 101 may include a field-programmable gate array (FPGA) in which the specific process or all processes are programmed. Furthermore, the control device 101 may include a solid state drive (SSD) as a memory. The control device 101 may include an SSD instead of the HDD 204 or may include both the HDD 204 and the SSD.

FIG. 3 is a diagram illustrating an example of a functional configuration of the control device 101. The control device 101 includes an examination controller 300, a signal obtaining unit 301, a position obtaining unit 302, the image processing unit 303, a determination unit 304, a display controller 305, and an output unit 306.

The examination controller 300 controls an examination performed by the imaging system 100. The examination controller 300 obtains information on an examination order from the ordering system 112. The examination order includes information on a patient to be examined and information on an imaging procedure. The examination controller 300 controls the probe 102 and the detection unit 103 based on the information on the imaging procedure. Furthermore, the examination controller 300 causes the display controller 305 to display the information on an examination on the display unit 104 so as to display the information on an examination for the user. The information on an examination displayed on the display unit 104 includes information on a patient to be examined, the information on the imaging procedure included in the examination, and an image which has been generated after imaging is completed. Furthermore, the examination controller 300 transmits information on a progress of the examination to the ordering system 112. For example, when the user starts the examination, the examination controller 300 transmits information on the start to the ordering system 112, and when the imaging in the entire imaging procedure included in the examination is completed, the examination controller 300 transmits information on the completion to the ordering system 112.

Furthermore, the examination controller 300 obtains information on the probe 102 being used in the imaging. The information on the probe 102 includes information on a type of the probe 102, a center frequency, sensitivity, an acoustic focus, an electronic focus, and an observation depth. The user connects the probe 102 to a probe connector port (not illustrated) of the control device 101, for example, enables the probe 102 by performing an operation input on the control device 101, and inputs an imaging condition and the like. The examination controller 300 obtains information on the enabled probe 102. The examination controller 300 appropriately transmits the information on the probe 102 to the image processing unit 303, the determination unit 304, and the display controller 305. The examination controller 300 is an example of second obtaining means for obtaining information on a movement of the probe 102.

The signal obtaining unit 301 obtains an ultrasonic signal and a photoacoustic signal from the probe 102. Specifically, the signal obtaining unit 301 separately obtains an ultrasonic signal and a photoacoustic signal from the information obtained from the probe 102 based on the information supplied from the examination controller 300 and the position obtaining unit 302. For example, in a case where a timing when an ultrasonic signal is obtained and a timing when a photoacoustic signal is obtained are determined in the imaging procedure in the imaging, an ultrasonic signal and a photoacoustic signal are separately obtained from the information obtained from the probe 102 based on the information on the timings of the obtainment obtained from the examination controller 300. As described in an example below, in a case where a photoacoustic signal is to be obtained based on information on a movement of the probe 102, an ultrasonic signal and a photoacoustic signal are separately obtained from the information obtained from the probe 102 based on the information on a movement of the probe 102 obtained from the position obtaining unit 302. The signal obtaining unit 301 is an example of first obtaining means which at least obtains one of an ultrasonic signal and a photoacoustic signal from the probe 102.

The position obtaining unit 302 obtains information on displacement of the probe 102 based on the information supplied from the detection unit 103. For example, the position obtaining unit 302 obtains at least one of information on a position of the probe 102, information on an orientation of the probe 102, information on a movement speed relative to the object, information on a rotation speed, information on acceleration of a movement relative to the object, and information on a degree of pressure relative to the object, based on the information supplied from the detection unit 103. Specifically, the position obtaining unit 302 obtains information on a user operation performed on the probe 102 relative to the object. The position obtaining unit 302 may determine whether the user stops the probe 102 in a state in which the probe 102 is in contact with the object or the user moves the probe 102 at a predetermined speed or more based on the information supplied from the position obtaining unit 302. The position obtaining unit 302 preferably obtains positional information of the probe 102 at a predetermined time interval from the detection unit 103 in real time.

The position obtaining unit 302 appropriately transmits the information on displacement of the probe 102 to the examination controller 300, the image processing unit 303, the determination unit 304, and the display controller 305. The position obtaining unit 302 is an example of second obtaining means which obtains information on displacement of the probe 102.

The image processing unit 303 generates an ultrasonic image, a photoacoustic image, and a superimposed image by superimposing a photoacoustic image on an ultrasonic image. The image processing unit 303 generates an ultrasonic image to be displayed on the display unit 104 using the ultrasonic signal obtained by the signal obtaining unit 301. The image processing unit 303 generates an ultrasonic image suitable for a set mode based on the information on the imaging procedure obtained from the examination controller 300. In a case where a Doppler mode is set as the imaging procedure, for example, the image processing unit 303 generates an image indicating a flow speed in the object based on a difference between a frequency of the ultrasonic signal obtained by the signal obtaining unit 301 and a transmission frequency.

Furthermore, the image processing unit 303 generates a photoacoustic image based on the photoacoustic signal obtained by the signal obtaining unit 301. The image processing unit 303 reconfigures a distribution of acoustic waves at a time when light is emitted based on the photoacoustic signal (hereinafter referred to as an initial acoustic pressure distribution). The image processing unit 303 divides the reconfigured the initial acoustic pressure distribution by light fluence distribution of the object relative to light emitted to the object so as to obtain an optical absorption coefficient distribution in the object. Furthermore, the image processing unit 303 obtains a concentration distribution of a substance in the object from the absorption coefficient distribution relative to a plurality of wavelengths utilizing a fact that a degree of absorption of light in the object varies depending on a wavelength of the light emitted to the object. For example, the image processing unit 303 obtains substance concentration distributions of oxyhemoglobin and deoxyhemoglobin in the object. Furthermore, the image processing unit 303 obtains an oxygen saturation distribution as a rate of oxyhemoglobin concentration to deoxyhemoglobin concentration. A photoacoustic image generated by the image processing unit 303 indicates information including the initial acoustic pressure distribution, the light fluence distribution, the absorption coefficient distribution, the substance concentration distribution, or the oxygen saturation distribution described above, for example. Specifically, the image processing unit 303 is an example of generation means for generating an ultrasonic image based on an ultrasonic signal and generates a photoacoustic image based on a photoacoustic signal.

The determination unit 304 determines whether the photoacoustic image is to be displayed on the display unit 104 through the display controller 305 based on the information on displacement of the probe 102 obtained by the position obtaining unit 302. Specifically, the determination unit 304 is an example of determination means for determining whether a photoacoustic image is to be displayed on the display unit 104.

The determination unit 304 determines that a photoacoustic image is to be displayed in a case where the position obtaining unit 302 obtains information indicating that the probe 102 is moving at a speed equal to or lower than a predetermined speed or in a case where the position obtaining unit 302 obtains information indicating that the probe 102 is pressed on the object in a predetermined pressure or more, for example. By this, the photoacoustic image is displayed on the display unit 104 when the user performs an operation to observe a specific region of the object. The user may observe the ultrasonic image and the photoacoustic image without a special operation input, such as a press of a switch having a physical structure.

In a case where the determination unit 304 determines that a photoacoustic image is to be displayed on the display unit 104, the image processing unit 303 generates a superimposed image by superimposing the photoacoustic image on the ultrasonic image and the superimposed image is displayed on the display unit 104 through the display controller 305, for example. Specifically, a mode for displaying the ultrasonic image is switched to a mode for displaying the ultrasonic image and the photoacoustic image. As another example, when the determination unit 304 determines that a photoacoustic image is to be displayed on the display unit 104, the examination controller 300 controls the irradiation unit 107 and the signal obtaining unit 301 so that a photoacoustic signal is obtained. Then the image processing unit 303 performs a reconfiguration process based on the photoacoustic signal obtained in accordance with the determination so that a photoacoustic image is generated. The display controller 305 displays the generated photoacoustic image on the display unit 104. In this point of view, the examination controller 300 is an example of irradiation control means for controlling the irradiation unit 107 so that the irradiation unit 107 irradiates the object with light in a case where it is determined that a photoacoustic image is to be displayed on the display unit 104.

The display controller 305 instructs the display unit 104 to display information on the display unit 104. The display controller 305 causes the display unit 104 to display information in accordance with an input from the examination controller 300, the image processing unit 303, and the determination unit 304 and an input of a user operation through the operation unit 105. The display controller 305 is an example of display control means. Furthermore, the display controller 305 is an example of display control means for displaying a photoacoustic image on the display unit 104 based on a result of a determination indicating that a photoacoustic image is to be displayed performed by the display controller 305.

The output unit 306 outputs information to an external apparatus, such as the PACS 113, through the network 110 from the control device 101. For example, the output unit 306 outputs the ultrasonic image, the photoacoustic image, and the superimposed image of the ultrasonic image and the photoacoustic image generated in the image processing unit 303 to the PACS 113. An image output from the output unit 306 includes a supplemental information attached by the examination controller 300 as various tags based on the DICOM standard. The supplemental information includes patient information, information indicating an imaging apparatus which has captured the image, an image ID for uniquely identifying the image, and an examination ID for uniquely identifying an examination in which the image is captured. Furthermore, the supplemental information includes information for associating the ultrasonic image and the photoacoustic image captured in a series of operations of the probe. The information for associating the ultrasonic image with the photoacoustic image indicates a frame which is closest to a timing when the photoacoustic image is obtained in a plurality of frames included in the photoacoustic image, for example. Furthermore, as the supplemental information, the positional information of the probe 102 obtained by the detection unit 103 may be attached to the frames of the ultrasonic image and the photoacoustic image. Specifically, the output unit 306 attaches information indicating a position of the probe 102 which has obtained the ultrasonic signal for generating the ultrasonic image to the ultrasonic image to be output. Furthermore, the output unit 306 attaches information indicating a position of the probe 102 which has obtained the photoacoustic signal for generating the photoacoustic image to the photoacoustic image to be output. The output unit 306 is an example of output means.

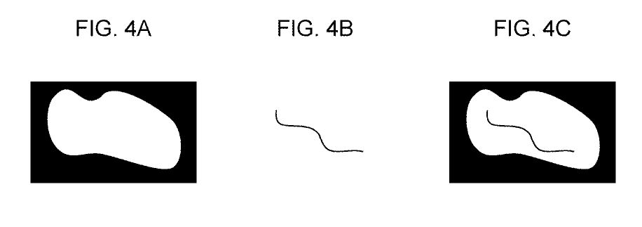

FIG. 4 includes diagrams illustrating examples of the ultrasonic image, the photoacoustic image, and the superimposed image, respectively, which are displayed on the display unit 104 by the display controller 305. FIG. 4A is a diagram illustrating an example of the ultrasonic image which is a tomographic image indicating amplitude of a reflection wave by luminance, that is, an example of an image generated in a B mode. Hereinafter, although a case where a B-mode image is generated as an ultrasonic image is illustrated as an example, an ultrasonic image obtained by the control device 101 in the first embodiment is not limited to a B-mode image. The obtained ultrasonic image may be generated in other methods, such as an A mode, an M mode, or a Doppler mode, or may be a harmonic image or a tissue elastic image. A region in the object which is to be captured as an ultrasonic image by the imaging system 100 is a region of circulatory organs, a breast, a liver, a pancreas, or the like. Furthermore, the imaging system 100 may capture an ultrasonic image of the object to which an ultrasonic contrast agent using microbubbles is administered, for example.

FIG. 4B is a diagram illustrating an example of the photoacoustic image which is an image of a blood vessel rendered based on the absorption coefficient distribution and the hemoglobin concentration. The photoacoustic image obtained by the control device 101 in the first embodiment may be any one of information on generated acoustic pressure (initial acoustic pressure) of a photoacoustic wave, information on optical absorption energy density, information on an optical absorption coefficient, information on concentration of a substance included in the object, and an image generated by combining the information. Furthermore, a region in the test image which is captured as a photoacoustic image by the imaging system 100 is a region of circulatory organs, a breast, an inguinal region, an abdomen, four extremities including fingers and toes, and the like. In particular, the blood vessel region including a new blood vessel and plaque on a blood vessel wall may be set as a target of the imaging of a photoacoustic image in accordance with characteristics associated with the optical absorption in the object. Although a case where a photoacoustic image is captured while an ultrasonic image is captured is illustrated as an example hereinafter, a region in the object captured as a photoacoustic image by the imaging system 100 may not correspond to a region captured as an ultrasonic image. Furthermore, the imaging system 100 may capture a photoacoustic image of the object to which a contrast agent including pigment, such as methylene blue or indocyanine green, gold fine particles, a substance obtained by collecting pigment and gold fine particles, or a substance obtained by chemically modifying pigment and gold fine particles is administered as a contrast agent.

FIG. 4C is a diagram illustrating a superimposed image obtained by superimposing the photoacoustic image on the ultrasonic image illustrated in FIGS. 4B and 4A, respectively. The image processing unit 303 generates the superimposed image by positioning the ultrasonic image and the photoacoustic image. The image processing unit 303 may use any method as a positioning method. For example, the image processing unit 303 performs the positioning based on a characteristic region which is rendered commonly in the ultrasonic image and the photoacoustic image. As another example, the image processing unit 303 may generate the superimposed image by superimposing the ultrasonic image and the photoacoustic image which have been determined to be rendered based on signals output from substantially the same region of the object based on information on the position of the probe 102 obtained by the position obtaining unit 302.

FIG. 5 is a diagram illustrating an example of a configuration of the imaging system 100. The imaging system 100 includes the console 501, the probe 102, the magnetic sensor 502, the magnetic transmitter 503, and a cradle 504. The console 501 is configured by integrating the control device 101, the display unit 104, and the operation unit 105. The control device according to the first embodiment is the control device 101 or the console 501. The magnetic sensor 502 and the magnetic transmitter 503 are examples of the detection unit 103. The cradle 504 supports the object.

The magnetic sensor 502 and the magnetic transmitter 503 are devices for obtaining positional information of the probe 102. The magnetic sensor 502 is a magnetic sensor attached to the probe 102. Furthermore, the magnetic transmitter 503 is disposed in an arbitrary position and forms a magnetic field outward from the magnetic transmitter 503 at a center. According to the first embodiment, the magnetic transmitter 503 is disposed in the vicinity of the cradle 504.

The magnetic sensor 502 detects a 3D magnetic field formed by the magnetic transmitter 503. Then the magnetic sensor 502 obtains positions (coordinates) of a plurality of points of the probe 102 in a space including the magnetic transmitter 503 as an origin based on information on the detected magnetic field. The position obtaining unit 302 obtains the 3D positional information of the probe 102 based on the information on the positions (coordinates) obtained from the magnetic sensor 502. The 3D positional information of the probe 102 includes a coordinate of the transmission/reception unit 106. The position obtaining unit 302 obtains a position of a plane which is in contact with the object based on the coordinate of the transmission/reception unit 106. Furthermore, the 3D positional information of the probe 102 includes information on an inclination (an angle) of the probe 102 relative to the object. Then the position obtaining unit 302 obtains information on displacement of the probe 102 based on a temporal change of the 3D positional information.

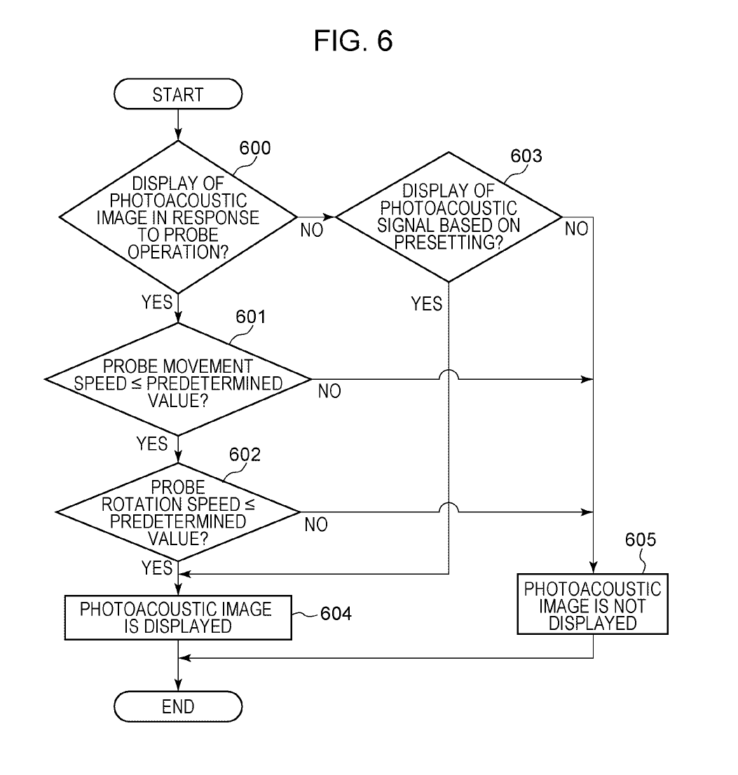

FIG. 6 is a flowchart of an example of a process of displaying a photoacoustic image on the display unit 104 based on a user operation performed on the probe 102 by the control device 101 according to the first embodiment. Hereinafter, a case where the user at least obtains an ultrasonic signal using the probe 102, operates the probe 102 while an ultrasonic image is displayed on the display unit 104, and displays a photoacoustic image on the display unit 104 will be described as an example.

In step S600, the examination controller 300 obtains information on a presetting associated with display of a photoacoustic image. The user performs the setting associated with display of a photoacoustic image by an operation input on the console 501 before examination. The setting associated with display of a photoacoustic image includes a setting associated with an obtainment of a photoacoustic signal and a setting associated with display of a photoacoustic image generated based on the obtained photoacoustic signal. According to the setting associated with an obtainment of a photoacoustic signal, a mode for operating the probe 102 is selected from among a first obtainment mode of obtaining an ultrasonic signal and a photoacoustic signal at predetermined timings, a second obtainment mode of obtaining a photoacoustic signal in accordance with a user operation performed on the probe 102 while an ultrasonic signal is obtained, and a third obtainment mode of obtaining only an ultrasonic signal. The first obtainment mode includes a case where an ultrasonic signal and a photoacoustic signal are alternately obtained by each predetermined period of time and a case where an ultrasonic signal and a photoacoustic signal are obtained in a mode determined in the order information obtained from the ordering system 112. The setting associated with display of a photoacoustic image includes a first display mode of successively displaying a photoacoustic image every time the photoacoustic image is reconfigured using a photoacoustic signal and a second display mode of not displaying a photoacoustic image until imaging is completed even when reconfiguration of a photoacoustic signal is performed. In a case where the setting associated with an obtainment of a photoacoustic signal is the second obtainment mode and the setting associated with display of a photoacoustic image is the first display mode, the process proceeds to step S601, and otherwise, the process proceeds to step S603.

In step S601, the determination unit 304 determines whether a movement speed of the probe 102 is equal to or lower than a predetermined value. Specifically, first, the position obtaining unit 302 obtains information on a position of the probe 102 from the magnetic sensor 502 and obtains information on a movement speed of the probe 102 based on a temporal change of the position. The position obtaining unit 302 transmits the information on the movement speed of the probe 102 to the determination unit 304. The determination unit 304 determines whether the movement speed of the probe 102 is equal to or lower than a predetermined value. Even when the probe 102 is stopped relative to the object, that is, the movement speed is zero, it is determined that the probe 102 moves at a speed lower than the predetermined speed. For example, the position obtaining unit 302 temporarily stores positional information of the probe 102 obtained by the magnetic sensor 502. Then the position obtaining unit 302 obtains a speed vector associated with the movement of the probe 102 and transmits the speed vector to the determination unit 304. The determination unit 304 determines that the position of the probe 102 is not sufficiently changed when the speed of the probe 102 is equal to or lower than the predetermined value for a predetermined period of time. For example, the determination unit 304 determines that a movement speed of the probe 102 is equal to or lower than the predetermined value when the probe 102 moves at a speed equal to or lower than the predetermined value for three seconds. The predetermined value is 50 mm/seconds. When the movement speed of the probe 102 is equal to or lower than the predetermined value, the process proceeds to step S602, and when the movement speed of the probe 102 is higher than the predetermined value, the process proceeds to step S605.

In step S602, the determination unit 304 determines whether a rotation speed of the probe 102 is equal to or smaller than a predetermined value. Specifically, as with step S601, first, the position obtaining unit 302 obtains information on a position of the probe 102 from the magnetic sensor 502 and obtains information on a rotation speed of the probe 102 based on a temporal change of the position. The position obtaining unit 302 transmits the information on the rotation speed of the probe 102 to the determination unit 304. The determination unit 304 determines whether the rotation speed of the probe 102 is equal to or lower than the predetermined value. Even when the probe 102 is stopped relative to the object, that is, the rotation speed is zero, the determination unit 304 determines that the probe 102 rotates at a speed lower than the predetermined speed. As with step S601, the position obtaining unit 302 obtains a speed vector associated with the movement of the probe 102 and transmits the speed vector to the determination unit 304. For example, the determination unit 304 determines that a rotation speed of the probe 102 is equal to or lower than the predetermined value when the probe 102 rotates at a speed equal to or lower than the predetermined value for three seconds. The predetermined value is ⅙ πrad/seconds. When the probe 102 rotates at a speed lower than the predetermined speed, the process proceeds to step S604. When the probe 102 rotates at a speed higher than the predetermined speed, the process proceeds to step S605.

In step S603, the process is branched based on the information on the presetting obtained by the examination controller 300 in step S600. In a case where the setting associated with an obtainment of a photoacoustic signal is the first obtainment mode and the setting associated with display of a photoacoustic image is the first display mode, the process proceeds to step S604, and otherwise, the process proceeds to step S605.

In step S604, the display controller 305 displays the photoacoustic image on the display unit 104. Specifically, the image processing unit 303 reconfigures the photoacoustic image based on a photoacoustic signal appropriately obtained based on the information on displacement of the probe 102 or a photoacoustic signal obtained at the predetermined timing. Then the display controller 305 displays the photoacoustic image on the display unit 104. According to the first embodiment, the image processing unit 303 generates a superimposed image by superimposing a photoacoustic image on an ultrasonic signal generated based on an ultrasonic signal obtained at a time point close to a time point when a photoacoustic signal is obtained. Then the display controller 305 displays the superimposed image on the display unit 104. Specifically, the display controller 305 displays the photoacoustic image generated from the photoacoustic signal based on the information on the displacement of the probe 102 on the display unit 104.

When the first obtainment mode is set, the user obtains the ultrasonic signal using the probe 102 and operates the probe 102 while observing the ultrasonic image displayed on the display unit 104. In a case where the movement speed or the rotation speed of the probe 102 is lower than the predetermined value, it is assumed that the user intends to observe a specific region in the object in detail. According to the first embodiment, the photoacoustic image is displayed on the display unit 104 in accordance with such a change of the user operation on the probe 102. Accordingly, the photoacoustic image may be displayed on the display unit 104 at an appropriate timing without disturbing the user observing the ultrasonic image to search for a region to be observed in detail. Furthermore, the display controller 305 may display the photoacoustic image included in the superimposed image in a higher transparent manner as the movement speed of the probe 102 is increased. When the movement speed of the probe 102 becomes higher than the predetermined value, the photoacoustic image may not be displayed. Specifically, the display controller 305 differentiates a display mode of the photoacoustic image on the display unit 104 in accordance with a degree of the displacement of the probe 102.

In step S605, the display controller 305 does not display the photoacoustic image on the display unit 104. The image processing unit 303 generates an ultrasonic image based on the ultrasonic signal obtained by the probe 102 and the display controller 305 displays the ultrasonic image on the display unit 104.

The process in FIG. 6 is thus terminated. Note that, although the case where the photoacoustic image is displayed on the display unit 104 in accordance with an operation on the probe 102 or the presetting is described as an example with reference to FIG. 6, the present invention is not limited to the display of a photoacoustic image. For example, the superimposed image or the photoacoustic image generated by the image processing unit 303 may be stored simultaneously with the display of the photoacoustic image on the display unit 104 in accordance with the operation of the probe 102. The storage is not limited to storage in a memory included in the control device 101, and the image may be output to an external apparatus, such as the PACS 113, through the output unit 306 and stored in the external apparatus. It is assumed that the user is searching for a region to be observed in detail while operating the probe 102 in a case where it is determined that the photoacoustic image may not be displayed according to the process in step S600 and step S603. Accordingly, such a moving image which is being searched for may not be stored. Therefore, the user may selectively store an image to be observed in detail by storing a superimposed image when it is determined that a photoacoustic image is to be displayed, and capacity of a memory and an external apparatus may be effectively utilized.

Note that the operations in step S601 and step S602 may be processed at the same time or in parallel. Specifically, the position obtaining unit 302 may transmit, at the same time or in parallel, information on the movement speed and the rotation speed of the probe 102 to the determination unit 304 based on the information indicating a position of the probe 102 obtained from the magnetic sensor 502. Then the determination unit 304 determines whether the movement speed of the probe 102 is equal to or lower than the predetermined value and the rotation speed is equal to or lower than the predetermined value. When the movement speed of the probe 102 is equal to or lower than the predetermined value and the rotation speed is equal to or lower than the predetermined value, the process proceeds to step S604. When at least one of the movement speed and the rotation speed of the probe 102 is equal to or higher than the predetermined value, the process proceeds to step S605. Furthermore, in another example, only one of the operations in step S601 and step S602 may be processed. Specifically, the determination unit 304 may make a determination as to whether a photoacoustic image is to be displayed based on one of the movement speed and the rotation speed.

### Modification of First Embodiment

According to the first embodiment, information for guiding the probe 102 to a region in which a photoacoustic signal of the object is to be obtained may be further displayed on a display unit 104. The information for guiding is used to guide a position of the probe 102 and an inclination of the probe 102 relative to the object to a target state. Specifically, first, in the second obtainment mode, the position obtaining unit 302 obtains positional information of the probe 102 based on positional information supplied from the detection unit 103.

The determination unit 304 stores the positional information of the probe 102 obtained when it is determined that a photoacoustic image is to be displayed on the display unit 104 during an operation on the probe 102. Hereinafter, a position of the probe 102 obtained when a preceding photoacoustic image is displayed is referred to as a target position. The determination unit 304 obtains positional information of the probe 102 from the position obtaining unit 302 as described above in the description of the process in step S602 and step S603, for example. The determination unit 304 generates guide information for guiding the probe 102 to the target position based on the target position and a current position of the probe 102. The guide information includes information on a movement direction, a movement amount, an inclination angle, a rotation direction, and a rotation amount obtained to move the probe 102 to the target position. In this point of view, the determination unit 304 is an example of guide means for generating guide information for guiding the probe 102 to a specific position.

For example, in a case where an operation of not determining that a photoacoustic image is to be displayed is performed although the probe 102 is operated near the target position for a predetermined period of time or more, the determination unit 304 generates the guide information. By this, a photoacoustic image and an ultrasonic image corresponding to a region observed by the user in detail in the observation may be easily reproduced.

The display controller 305 displays the guide information generated by the determination unit 304 on the display unit 104. Specifically, the display controller 305 displays a guide image serving as an objective index indicating a movement direction, a movement amount, an inclination angle, a rotation direction, and a rotation amount for moving the probe 102 to the target position on the display unit 104. Any guide image may be employed as long as the guide image serves as an objective index for the guide information. For example, the guide image corresponds to an image of an arrow mark having a size corresponding to an amount of a movement or a rotation and having a direction corresponding to a direction of the movement, the rotation, or an inclination. As another example, the guide image is a graphic which has a size corresponding to an amount of a movement or a rotation and which has a shape deformed in accordance with a direction of a movement, a rotation, and an inclination. The guide image is displayed on the display unit 104 such that the observation on the region (hereinafter referred to as a target region) to be rendered in an ultrasonic image or a photoacoustic image is not disturbed when the probe 102 is moved to the target position. For example, the guide image is displayed in a region in which an ultrasonic image, a photoacoustic image, or a superimposed image is not displayed. As another example, while the probe 102 is guided to the target position, the guide image may be displayed in a position superimposed on a region in the vicinity of the target region and deformed to a form which is not visually recognized after the target region is rendered.

As a further example, a notification indicating the guide information generated by the determination unit 304 may be made for the user by generating a sound such that a sound generation interval is reduced as the probe 102 moves closer to the target position.

Note that the determination unit 304 may determine that the guide information is to be generated and causes the position obtaining unit 302 to generate the guide information, and thereafter, the position obtaining unit 302 may generate the guide information. Furthermore, the guide information may be generated by a module disposed separately from the position obtaining unit 302 and the determination unit 304.

Although the case where the position of the probe 102 in which the user may render the region observed in detail in the observation is stored for generation of guide information is described as an example in the foregoing example, the present invention is not limited to this. For example, a position of the probe 102 in which a region specified based on an ultrasonic image obtained during operation of the probe 102, an ultrasonic image observed in the past, a photoacoustic image, and other medical images may be rendered may be stored as a position of the probe 102 for generating the guide information. Although the case where the position of the probe 102 for generating the guide information is automatically stored when a determination as to whether a photoacoustic image is to be displayed is made is described as an example, the present invention is not limited to this and the user may specify the position by an operation input performed on the console 501.

Furthermore, although the case where the guide information for reproducing an image of a region which is observed by the user in detail in the observation is generated is illustrated as an example in the foregoing example, the present invention is not limited to this. For example, a case where a 3D photoacoustic image of a specific region is obtained in accordance with the examination order or an operation input by a user will be described. When a photoacoustic signal is obtained while the user operates the probe 102, a signal which is sufficient for generation of a 3D photoacoustic image is required to be obtained. The image processing unit 303 generates information on a signal which is required for generating a 3D photoacoustic image based on a photoacoustic signal transmitted from the signal obtaining unit 301 and positional information of the probe 102 transmitted from the position obtaining unit 302. The position obtaining unit 302 generates guide information for guiding the probe 102 to a position where the signal which is required for generating a 3D photoacoustic image and displays the guide information on the display unit 104 through the display controller 305. In this way, the 3D photoacoustic image may be efficiently generated.

Although the case where the magnetic sensor 502 and the magnetic transmitter 503 are used as an example of the detection unit 103 according to the first embodiment is described above, the present invention is not limited to this.

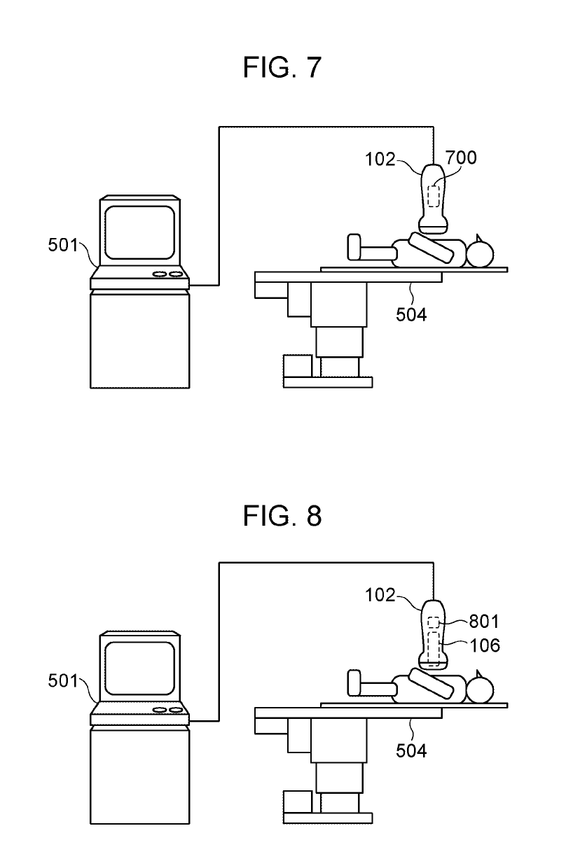

FIG. 7 is a diagram illustrating an example of a configuration of the imaging system 100. The imaging system 100 includes the console 501, the probe 102, the cradle 504, and a motion sensor 700. The motion sensor 700 is an example of the detection unit 103 which tracks positional information of the probe 102. The motion sensor 700 is disposed or embedded in a portion different from the transmission/reception unit 106 and the light source (not illustrated) of the probe 102. The motion sensor 700 is constituted by a micro electro mechanical systems, for example, and provides nine-axis motion sensing including a three-axis accelerometer, a three-axis gyroscope, and a three-axis magnetic compass. The position obtaining unit 302 obtains information on displacement of the probe 102 detected by the motion sensor 700.

### Second Embodiment

In a second embodiment, a case where a photoacoustic image is displayed on a display unit 104 in accordance with a pressure for pressing a probe 102 to an object will be described as an example. Only portions different from the first embodiment are described and descriptions of portions which are the same as those in the first embodiment are omitted since the foregoing descriptions are incorporated herein. A control device according to the second embodiment is a control device 101 and a console 501.

FIG. 8 is a diagram illustrating an example of a configuration of an imaging system 100. The imaging system 100 includes the console 501, the probe 102, a cradle 504, a transmission/reception unit 106, and a pressure sensor 801.

The pressure sensor 801 is an example of a detection unit 103. The pressure sensor 801 obtains information indicating a degree of pressure obtained when the user presses the probe 102 to the object as information on a mode of displacement of the probe 102. The transmission/reception unit 106 is disposed inside the probe 102 as a semifixed floating structure. The pressure sensor 801 is disposed on a surface which is opposite to a surface in which the transmission/reception unit 106 is in contact with the object and measures a pressure applied to the transmission/reception unit 106. Note that the pressure sensor 801 may be a diaphragm type pressure sensor disposed on a contact plane of the probe 102 relative to the object. The position obtaining unit 302 obtains information on pressure measured by the pressure sensor 801.

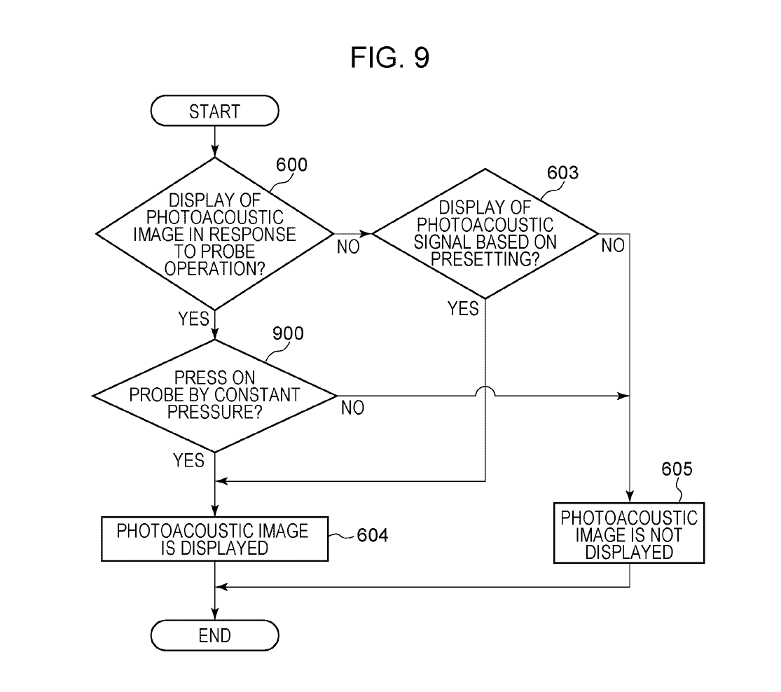

FIG. 9 is a flowchart of an example of a process of displaying a photoacoustic image on the display unit 104 based on a user operation on the probe 102 performed by the control device 101 according to the second embodiment. Hereinafter, a case where the user at least obtains an ultrasonic signal using the probe 102, operates the probe 102 while an ultrasonic image is displayed on the display unit 104, and displays a photoacoustic image on the display unit 104 will be described as an example. The processes in step S600, step S603, step S604, and step S605 are the same as those in the first embodiment which has been described with reference to FIG. 6.

In step S900, the determination unit 304 determines whether the user causes the probe 102 to press the object by a constant pressure. Specifically, the position obtaining unit 302 transmits information obtained from the pressure sensor 801 to the determination unit 304. When the pressure applied to the transmission/reception unit 106 is included in a predetermined range for a predetermined period of time or more, the determination unit 304 determines that the user presses the probe 102 onto the object by a constant pressure. When the user presses the probe 102 onto the object by the constant pressure, the process proceeds to step S604. When the user presses the probe 102 by the constant pressure, it is assumed that the user is observing a specific region of the object. By this, a photoacoustic image may be displayed on the display unit 104 in a case where the user desires to observe the specific region of the object. When the user does not press the probe 102 by the constant pressure, the process proceeds to step S605 and a photoacoustic image is not displayed.

In step S604, the image processing unit 303 generates a superimposed image by superimposing a photoacoustic image on an ultrasonic image, for example, and displays the superimposed image on the display unit 104. According to the second embodiment, furthermore, the image processing unit 303 may obtain information on the pressure from the position obtaining unit 302 and display a photoacoustic image on the display unit 104 based on the pressure information. As the user presses the probe 102 by the constant pressure for a longer period of time, it is assumed that it is highly likely that the user focuses on a region extracted at the time. Therefore, as a period of time in which a pressure value of the pressure sensor 801 is constant is longer, the image processing unit 303 sets lower transparency of the photoacoustic image in the superimposed image. Specifically, the display controller 305 differentiates a display mode of the photoacoustic image on the display unit 104 in accordance with a degree of the displacement of the probe 102. By this, the user may observe the photoacoustic image in accordance with a degree of attention.

Note that, although the case where a photoacoustic image is displayed on the display unit 104 based on a pressure for pressing the probe 102 onto the object has been described in the second embodiment, the present invention is not limited to this. The probe 102 may include a magnetic sensor 502 or a motion sensor 700. The determination unit 304 may determine whether a photoacoustic image is to be displayed based on information on a position of the probe 102 and an angle relative to the object instead of the pressure for pressing the probe 102 onto the object. Specifically, the display controller 305 may display a photoacoustic image on the display unit 104 when the position obtaining unit 302 obtains at least one of information indicating that the probe 102 moves at a speed lower than a predetermined speed relative to the object and information indicating that the probe 102 is pressed onto the object by a constant pressure.

### Third Embodiment

In a third embodiment, a case where a photoacoustic image is displayed on a display unit 104 in accordance with characteristics of a probe 102 used by a user for observation of an object and a purpose of an examination will be described as an example. Only portions different from the first embodiment are described and descriptions of portions which are the same as those in the first embodiment are omitted since the foregoing descriptions are incorporated. A control device according to the third embodiment is a control device 101 and a console 501.

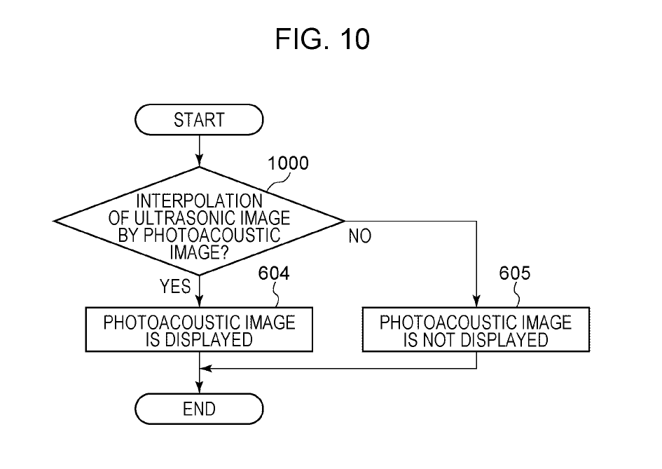

FIG. 10 is a flowchart of an example of a process of displaying a photoacoustic image in accordance with the characteristics of the probe 102 and the purpose of the examination performed by the control device according to the third embodiment. Hereinafter, a case where the user at least obtains an ultrasonic signal using the probe 102, operates the probe 102 while an ultrasonic image is displayed on a display unit 104, and further displays a photoacoustic image on the display unit 104 will be described as an example. A plurality of probes may be connected to the console 501 and the user selects one of the probes to be used in accordance with the purpose of the examination, such as a region for observing the object. The processes in step S604 and step S605 are the same as those in the first embodiment which is described with reference to FIG. 6.

In step S1000, a determination unit 304 determines whether an ultrasonic image may be interpolated by a photoacoustic image. Specifically, the examination controller 300 obtains an imaging condition of the ultrasonic image and the photoacoustic image and transmits the imaging condition to the determination unit 304. A position obtaining unit 302 obtains information on the probe 102 used by the user in the observation and transmits the information to the determination unit 304. The information on the probe 102 includes an array of transducers (not illustrated) of the probe 102, an initial setting when the probe is connected to the console 501, information on a scan method, and information indicating whether an irradiation unit 107 is included. When the determination unit 304 determines that the ultrasonic image may be interpolated by the photoacoustic image, the process proceeds to step S604. When the determination unit 304 determines that the ultrasonic image may not be interpolated by the photoacoustic image, the process proceeds to step S605.

Characteristics of the obtained ultrasonic image vary depending on the imaging condition including array of transducers, a scan method, and a setting for obtaining a signal. For example, an ultrasonic image of a wide field is obtained in a depth portion of the object when a convex electronic scan method is employed which is used in observation of an abdominal region. An ultrasonic image of a wide field is obtained from a narrow contact portion and is mainly used for observation of a circulatory organ region. Furthermore, when ultrasonic waves of a high frequency are used, an ultrasonic image of high resolution is obtained. However, transparency of the ultrasonic signal is low, and therefore, a region of the object rendered in the ultrasonic image is shallow. In this way, different characteristics are obtained in the ultrasonic images rendered in different imaging conditions, and therefore, the determination unit 304 determines whether an ultrasonic image is to be displayed on the display unit 104 in accordance with the characteristics. For example, in a case where a depth of the object rendered in the photoacoustic image is larger than a depth of the object rendered in the ultrasonic image, the determination unit 304 determines that the ultrasonic image may be interpolated by the photoacoustic image.

Furthermore, when an ultrasonic signal is obtained using ultrasonic waves of a middle frequency while priority is given to the depth of the object, resolution of the rendered ultrasonic image may be insufficient for detailed observation. Accordingly, it is assumed that additional observation of the photoacoustic image is effective for interpolating lack of the resolution. For example, when the resolution of the photoacoustic image is higher than that of the ultrasonic image, the determination unit 304 determines that the ultrasonic image may be interpolated by the photoacoustic image.

When the probe 102 does not include an irradiation unit 107 and is only used for an obtainment of an ultrasonic signal, a photoacoustic signal may not be obtained. Accordingly, the determination unit 304 determines that the ultrasonic image may not be interpolated by the photoacoustic image.

Specifically, the determination unit 304 determines whether the photoacoustic image is to be displayed on the display unit 104 based on the characteristics of the probe 102 used for the observation. Characteristics of a rendered ultrasonic image and characteristics of a photoacoustic image both depend on the characteristics of the probe 102. Accordingly, the determination unit 304 makes the determination based on the characteristics of the probe 102 including an imaging condition and a configuration of the probe 102 which are associated with the characteristics of the ultrasonic image and the photoacoustic image. Furthermore, the position obtaining unit 302 which obtains information on the characteristics of the probe 102 is an example of third obtaining means for obtaining the information on the characteristics of the ultrasonic image rendered based on the ultrasonic signal obtained by the probe 102.

In the foregoing example, the case where an ultrasonic image is interpolated by a photoacoustic image in accordance with a depth or resolution of the object rendered in an image is described as an example. A criterion for the determination as to whether an ultrasonic image is interpolated by a photoacoustic image may be appropriately set by the user by specifying a parameter of the depth or the resolution.

In the foregoing example, although the case where it is determined whether a photoacoustic image is to be displayed on the display unit 104 is described as an example, the present invention is not limited to this. A superimposed image may be displayed by superimposing a photoacoustic image only on a portion of a region of the object displayed on the display unit 104. By this, the photoacoustic image is not superimposed on the region in which a structure of the object is rendered in detail in the ultrasonic image, and therefore, observation of the ultrasonic image is not disturbed. As for a region in which the structure of the object is not rendered in detail in the ultrasonic image, observation of the region by the user may be assisted by superimposing the photoacoustic image. The transparency of the superimposed photoacoustic image may be differentiated depending on a degree of the depth or the resolution described above.

In the foregoing example, the case where it is determined whether the photoacoustic image is to be displayed based on a parameter of the ultrasonic image is described as an example. A determination as to whether a photoacoustic image is to be displayed may be made in advance for each of the plurality of probes connected to the console 501.

The probe 102 according to the third embodiment may include a magnetic sensor 502 and a motion sensor 700. The determination unit 304 may determine whether a photoacoustic image is to be displayed based on information on the pressure for pressing the probe 102 onto the object, information on a position of the probe 102, or information on an angle relative to the object instead of the parameter of the ultrasonic image.

Furthermore, when the probe 102 which is not suitable for examination order obtained by the ordering system 112 is used, a notification indicating that the probe being used is not appropriate may be made for the user. For example, a message or an image indicating an inappropriate probe is displayed on the display unit 104 as the notification. Alternatively, an obtainment of a photoacoustic signal may be disabled and a notification indicating that the disabling may be made for the user. Examples of the inappropriate case include a case where a probe which does not include the irradiation unit 107 for obtaining a photoacoustic signal is used irrespective of a request for obtaining a photoacoustic signal in accordance with examination order.

### Fourth Embodiment

Although the case where a photoacoustic image generated by the image processing unit 303 is displayed on the display unit 104 is illustrated in the first to third embodiments, the present invention is not limited to this. For example, in a case where it is determined that the determination unit 304 determines the display on the display unit 104 as described above, an examination controller 300 may control the irradiation unit 107 so as to obtain a photoacoustic signal. Thereafter, a photoacoustic image reconfigured based on a photoacoustic signal obtained in accordance with the determination may be displayed on the display unit 104.

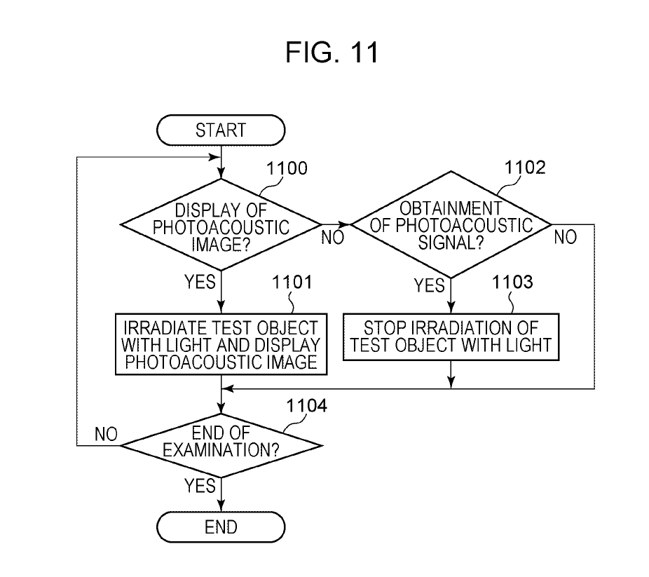

FIG. 11 is a flowchart of an example of a process of controlling the irradiation unit 107 based on a determination performed by the determination unit 304, obtaining a photoacoustic image, and displaying the photoacoustic image on the display unit 104.

In step S1100, the determination unit 304 determines whether a photoacoustic image is to be displayed on the display unit 104. Step S1100 corresponds to the process in step S600 and step S603 according to the first embodiment, the process in step S600, step S603, and step S900 according to the second embodiment, and step S1000 according to the third embodiment. When it is determined that the display is performed, the process proceeds to step S1101, and when it is determined that the display is not performed, the process proceeds to step S1102.

In step S1101, the examination controller 300 instructs the irradiation unit 107 to irradiate an object with light. The signal obtaining unit 301 obtains a photoacoustic signal from the probe 102. The image processing unit 303 reconfigures the photoacoustic image using the photoacoustic signal. The display controller 305 displays the photoacoustic image on the display unit 104. Step S1101 corresponds to step S604 according to the first to third embodiments.

In step S1102, the position obtaining unit 302 obtains information on a state of the probe 102. When information indicating that a photoacoustic signal is being obtained is obtained, the process proceeds to step S1103. When information indicating that a photoacoustic signal is not being obtained is obtained, the process proceeds to step S1104.

In step S1103, the examination controller 300 instructs the irradiation unit 107 to stop irradiating the object with light. The process in step S1102 and step S1103 corresponds to step S605 according to the first to third embodiments.

In step S1104, the examination controller 300 determines whether an examination for imaging an ultrasonic image and a photoacoustic image is to be terminated. For example, the user may instruct the end of the examination by an operation input on the console 501. Alternatively, the examination controller 300 may obtain positional information of the probe 102 from the position obtaining unit 302 and determines that the examination is to be terminated when a state in which the probe 102 is not in contact with the object is continued for a predetermined period of time. When it is determined that the examination is to be terminated based on the positional information, a screen for a determination as to whether the examination is to be terminated is preferably displayed for the user on the display unit 104 through the display controller 305. When an instruction for terminating the examination has not been detected, the process returns to step S1100, and when the instruction for terminating the examination has been detected, the process in FIG. 11 is terminated.

Accordingly, irradiation on the object with light may be controlled when a photoacoustic image is required to be displayed, and safety of the user and the object may be improved.

Note that the irradiation unit 107 is controlled by the signal obtaining unit 301, for example. The signal obtaining unit 301 preferably performs light irradiation in a period in which influence of a body motion caused by breathing or heartbeat is seen to be small and controls the various components in the irradiation unit 107 so as to obtain a photoacoustic signal. For example, the signal obtaining unit 301 may instruct the irradiation unit 107 to start light irradiation within 250 ms after it is determined that a photoacoustic image is to be displayed in step S1100. Furthermore, a period of time from when the determination is made to when the light irradiation is performed may be a predetermined value or may be specified by the user through the operation unit 105.

### First Modification

The case where the determination unit 304 determines whether a photoacoustic image is to be displayed on the display unit 104 is described as an example in the first to fourth embodiments. A process of displaying a photoacoustic image on the display unit 104 based on a determination made by the determination unit 304 is not limited to the foregoing example. The control device 101 may continuously obtain an ultrasonic signal and a photoacoustic signal and generate a photoacoustic image when it is determined that a photoacoustic image is to be displayed. Furthermore, the control device 101 may obtain a photoacoustic signal when it is determined that a photoacoustic image is to be displayed. Furthermore, a mode for displaying a photoacoustic image on the display unit 104 is not limited to the foregoing example. Display of an ultrasonic image on the display unit 104 may be switched to display of a photoacoustic image, an ultrasonic image and a photoacoustic image may be displayed in parallel, or a superimposed image obtained by superimposing a photoacoustic image on an ultrasonic image may be displayed.

The case where the determination unit 304 performs the determination based on information on displacement of the probe 102, that is, information indicating a user operation performed on the probe 102, is described as an example in the first to fourth embodiments. The determination made by the determination unit 304 is not limited to this. For example, the control device 101 may include a sound collecting microphone which receives an instruction issued by voice of the user. The control device 101 may store a voice recognition program to be executed so that an instruction issued by voice of the user is discriminated.

In addition to the first to fourth embodiments, a determination as to whether a photoacoustic image is to be displayed may be made based on a result of a determination as to whether a predetermined period of time has elapsed after a parameter of the probe 102 is controlled may be further made. It is assumed that the user controls parameters of sensitivity, focus, and a depth of the probe 102 by a user operation input to the console 501 or the probe 102. In this case, the determination unit 304 determines that a photoacoustic image is not to be displayed on the display unit 104 until a predetermined period of time has elapsed after the parameter control is performed. By this, if the user desires to continuously perform observation using the changed parameters, a photoacoustic image is displayed, and if the parameters are further likely to be changed, a photoacoustic image is not displayed. The user may easily control the parameters while observing an ultrasonic image, and a workflow may be improved.

Furthermore, a notification indicating that the probe 102 is irradiated with light may be made for the user in the first to fourth embodiments. For example, a notification image which notifies the user of the irradiation on the probe 102 with light is displayed on the display unit 104. In a case where the notification image is to be displayed on the display unit 104, the notification image is preferably displayed in a portion in the vicinity of an image of the object observed by the user. As another example, the probe 102 may include LED light which is lit during irradiation on the probe 102 with light. As a further example, the control device 101 may generate a notification sound during the irradiation with light. In this point of view, the display controller 305 which displays a guide image on the display unit 104, the LED light disposed in the probe 102, and a sound generator which generates the notification sound are examples of notification means for notifying the user of light irradiation performed to obtain a photoacoustic signal. Accordingly, even in a case where there is an interval between when the probe 102 is controlled so that a photoacoustic signal is obtained to when a photoacoustic image is displayed on the display unit 104, for example, the user may recognize that the probe 102 is irradiating the object with light and safety of the user and the object may be improved.

### Second Modification

In the foregoing embodiments, the case where a photoacoustic image is superimposed on an ultrasonic image is described as an example. In this modification, a method for not displaying a photoacoustic image which has been superimposed on an ultrasonic image will be described.

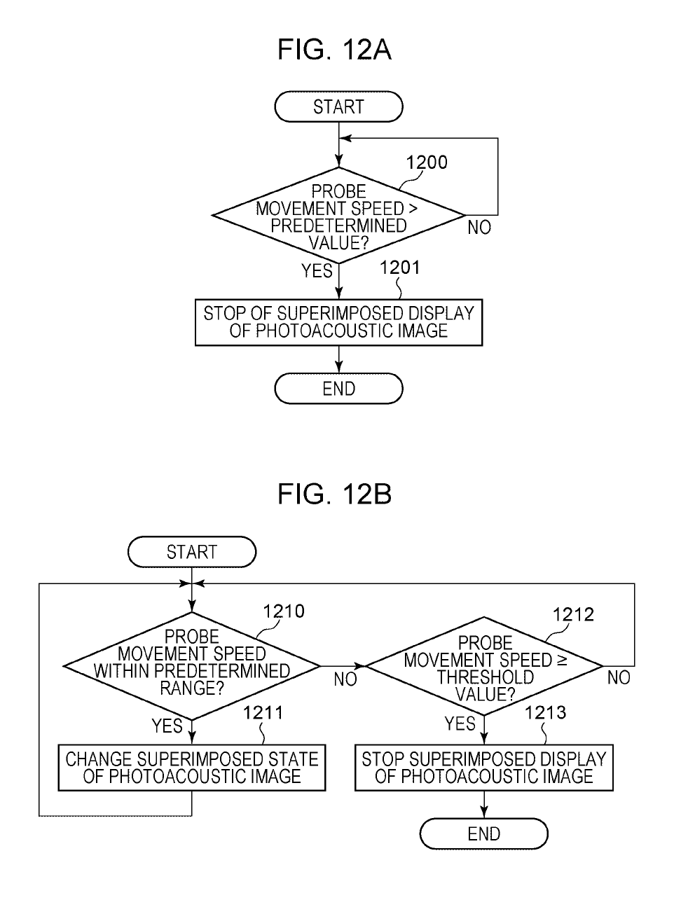

FIGS. 12A and 12B are flowcharts of examples of a process of stopping a superimposed display of a photoacoustic image which is superimposed on an ultrasonic image. First, an example of a method for not displaying a photoacoustic image superimposed on an ultrasonic image will be described with reference to FIG. 12A.

A process in step S1200 is executed after a photoacoustic image is displayed on an ultrasonic image. Specifically, this embodiment may be combined with an arbitrary one of the foregoing embodiments.

In step S1200, the determination unit 304 determines whether a movement speed of the probe 102 is higher than a predetermined value. Specifically, first, the position obtaining unit 302 obtains information on a position of the probe 102 from the magnetic sensor 502 and obtains information on a movement speed of the probe 102 based on a temporal change of the position. The position obtaining unit 302 transmits the information on the movement speed of the probe 102 to the determination unit 304.

The determination unit 304 obtains the information indicating the movement speed of the probe 102 transmitted from the position obtaining unit 302 and determines whether the movement speed of the probe 102 is higher than the predetermined value based on the obtained information indicating the movement speed of the probe 102. Here, the predetermined value is the same as the predetermined value used in step S601, for example. When the determination unit 304 determines that the movement speed of the probe 102 is higher than the predetermined value, the process proceeds to step S1201. Furthermore, when the determination unit 304 determines that the movement speed of the probe 102 is equal to or lower than the predetermined value, the process returns to step S1200 again.

Note that the determination unit 304 may determine that the movement speed of the probe 102 is higher than the predetermined value when a period of time in which the movement speed of the probe 102 is higher than the predetermined value is continued for a predetermined period of time.

In step S1201, the display controller 305 displays an ultrasonic image on which a photoacoustic image is not superimposed on the display unit 104 instead of the superimposed image displayed on the display unit 104. Specifically, the display controller 305 displays an ultrasonic image on which a photoacoustic image is not superimposed on the display unit 104 in real time.

According to the example of the process illustrated in FIG. 12A, in a case where an ultrasonic image on which a photoacoustic image is not superimposed is to be observed in detail, the user may display the desired ultrasonic image on the display unit by a simple operation performed on the probe 102.

Note that, although the superimposed display of the photoacoustic image is stopped using the movement speed of the probe 102 in the foregoing example, the display controller 305 may stop the superimposed display of the photoacoustic image using another information. For example, a rotation speed of the probe 102 may be used instead of the movement speed of the probe 102. Furthermore, the display controller 305 may stop the superimposed display of the photoacoustic image when the rotation speed of the probe 102 is higher than a predetermined value, for example. Note that the predetermined value to be compared with the rotation speed of the probe 102 is the same as the predetermined value used in step S602, for example.

Furthermore, the display controller 305 may stop the superimposed display of the photoacoustic image when the movement speed of the probe 102 and the rotation speed of the probe 102 are higher than the respective predetermined values.

Furthermore, acceleration of the probe 102 may be used instead of the movement speed of the probe 102. For example, the display controller 305 may stop the superimposed display of the photoacoustic image when the acceleration of the probe 102 is larger than a predetermined value.

Next, an example of a method for performing switching between display and non-display of a photoacoustic image superimposed on an ultrasonic image will be described with reference to FIG. 12B.

A process in step S1210 is executed after a photoacoustic image is displayed on an ultrasonic image. Specifically, this embodiment may be combined with arbitrary one of the foregoing embodiments.

In step S1210, the determination unit 304 determines whether a movement speed of the probe 102 is within a predetermined range. The determination unit 304 obtains the information indicating the movement speed of the probe 102 transmitted from the position obtaining unit 302 and determines whether the movement speed of the probe 102 is within the predetermined range based on the obtained information indicating the movement speed of the probe 102. Here, the predetermined range is a range larger than the predetermined value used in step S601 and smaller than another predetermined value, for example. When the determination unit 304 determines that the movement speed of the probe 102 is within the predetermined range, the process proceeds to step S1211. Furthermore, when the determination unit 304 determines that the movement speed of the probe 102 is out of the predetermined range, the process proceeds to step S1212 again.

Note that the determination unit 304 may determine that the movement speed of the probe 102 is higher than the predetermined value when a period of time in which the movement speed of the probe 102 is within the predetermined range is continued for a predetermined period of time.

In step S1211, the display controller 305 changes a superimposed state of the photoacoustic image. For example, in a case where a photoacoustic image is superimposed on an ultrasonic image before step S1211, the display controller 305 displays an ultrasonic image on which a photoacoustic image is not superimposed on the display unit 104 instead of the ultrasonic image displayed on the display unit 104 in step S1211. On the other hand, in a case where a photoacoustic image is not superimposed on an ultrasonic image before step S1211, the display controller 305 displays an ultrasonic image on which a photoacoustic image is superimposed on the display unit 104 instead of the ultrasonic image displayed on the display unit 104 in step S1211. Specifically, in step S1211, switching of a superimposed state of a photoacoustic image is executed. Note that the determination unit 304 may not execute the determination in step S1210 within a predetermined period of time after the superimposed state is changed in step S1211 so that the superimposed state is not frequently changed. The same is true on other examples described below.

In step S1212, the determination unit 304 determines whether the movement speed of the probe 102 is equal to or higher than another predetermined value (a threshold value) which is an upper limit of the predetermined range. When the determination unit 304 determines that the movement speed of the probe 102 is equal to or higher than the threshold value, the process proceeds to step S1213. When the determination unit 304 determines that the movement speed of the probe 102 is lower than the threshold speed (that is, the movement speed is equal to or smaller than the predetermined value used in step S601), the process returns to step S1210 again. Specifically, according to the example of the process illustrated in FIG. 12B, when the superimposed state of the photoacoustic image is changed once, a display state is maintained even if the probe is stopped.

In step S1213, the display controller 305 displays an ultrasonic image on which a photoacoustic image is not superimposed on the display unit 104 instead of the superimposed image displayed on the display unit 104. Specifically, the display controller 305 displays an ultrasonic image on which a photoacoustic image is not superimposed on the display unit 104 in real time. Note that, in a case where a photoacoustic image is not superimposed on an ultrasonic image before step S1213, the display controller 305 continuously displays the ultrasonic image on which a photoacoustic image is not superimposed on the display unit 104.

As an example of the process illustrated in FIG. 12B, a result of the determination as to whether a photoacoustic image is superimposed on an ultrasonic image may be switched by a simple operation performed on the probe 102. Accordingly, the user may observe an ultrasonic image on which a photoacoustic image is not superimposed in detail by a simple operation performed on the probe 102. Furthermore, the user may superimpose a photoacoustic image on an ultrasonic image again by a simple operation performed on the probe 102.

Note that, although the case where the superimposed state of the photoacoustic image is changed using the movement speed of the probe 102 in the foregoing example, the display controller 305 may change the superimposed state of the photoacoustic image using another information. For example, the rotation speed of the probe 102 may be used instead of the movement speed of the probe 102. Furthermore, the display controller 305 may change the superimposed state of the photoacoustic image when the rotation speed of the probe 102 is within a predetermined range, for example.

Furthermore, the display controller 305 may stop the superimposed state of the photoacoustic image when the movement speed of the probe 102 and the rotation speed of the probe 102 are within the respective predetermined ranges.

Furthermore, acceleration of the probe 102 may be used instead of the movement speed of the probe 102. Furthermore, the display controller 305 may change the superimposed state of the photoacoustic image when acceleration of the probe 102 is within a predetermined range, for example.

Furthermore, although the display controller 305 superimposes a photoacoustic image on an ultrasonic image in accordance with the movement speed of the probe 102 according to the first embodiment, a pressure applied to the probe 102 toward the object may be further used. For example, the display controller 305 may display a photoacoustic image superimposed on an ultrasonic image on the display unit 104 in a case where the movement speed of the probe 102 is equal to or lower than the predetermined value and a pressure applied to the probe 102 so that the probe 102 presses the object is equal to or larger than a predetermined value. The display controller 305 changes the superimposed state of the photoacoustic image in a case where, in a state in which the photoacoustic image and the ultrasonic image are displayed on the display unit 104 such that the photoacoustic image is superposed on the ultrasonic image, when the movement speed of the probe 102 is higher than the predetermined value and a pressure applied to the probe 102 pressing the object is equal to or larger than the predetermined value, the display controller 305 changes the superimposed state of the photoacoustic image. Specifically, in a case where a photoacoustic image is superimposed on an ultrasonic image in advance, the display controller 305 displays an ultrasonic image on which a photoacoustic image is not superimposed on the display unit 104 instead of a superimposed image displayed on the display unit 104. Furthermore, in a case where a photoacoustic image is not superimposed on an ultrasonic image in advance, the display controller 305 displays an ultrasonic image on which a photoacoustic image is not superimposed on the display unit 104 instead of a ultrasonic image displayed on the display unit 104.

Note that the pressure applied to the probe 102 toward the object is smaller than a predetermined value, the display controller 305 displays an ultrasonic image on which a photoacoustic image is not displayed on the display unit 104.

Also by the process described above, a result of a determination as to whether a photoacoustic image is to be superimposed on an ultrasonic image may be switched by a simple operation performed on the probe 102. Accordingly, the user may observe an ultrasonic image on which a photoacoustic image is not superimposed in detail by a simple operation performed on the probe 102. Furthermore, the user may superimpose a photoacoustic image on an ultrasonic image again by a simple operation performed on the probe 102.

Furthermore, according to the first embodiment, the display controller 305 displays a photoacoustic image superimposed on an ultrasonic image on the display unit 104 when the movement speed of the probe 102 is equal to or lower than the predetermined value. In this case, the display controller 305 may change the superimposed state of the photoacoustic image based on information indicating an angle of the probe 102 detected by a gyroscope sensor. For example, the display controller 305 changes the superimposed state of the photoacoustic image when the movement speed of the probe 102 is equal to or lower than the predetermined value and a change of an angle of the probe 102 in a predetermined period of time is equal to or larger than a predetermined value. Specifically, the display controller 305 changes the superimposed state of the photoacoustic image when the user intends to change only the angle without changing a position of a tip of the probe 102, for example. Accordingly, in a case where a photoacoustic image is superimposed on an ultrasonic image in advance, the display controller 305 displays an ultrasonic image on which a photoacoustic image is not superimposed on the display unit 104 instead of a superimposed image displayed on the display unit 104. Furthermore, in a case where a photoacoustic image is not superimposed on an ultrasonic image in advance, the display controller 305 displays an ultrasonic image on which a photoacoustic image is superimposed on the display unit 104 instead of an ultrasonic image displayed on the display unit 104.

Note that the display controller 305 displays an ultrasonic image on which a photoacoustic image is not superimposed on the display unit 104 when the movement speed of the probe 102 becomes higher than the predetermined value.

Also by the process described above, a result of a determination as to whether a photoacoustic image is to be superimposed on an ultrasonic image may be switched by a simple operation performed on the probe 102. Accordingly, the user may observe an ultrasonic image on which a photoacoustic image is not superimposed in detail by a simple operation performed on the probe 102. Furthermore, the user may superimpose a photoacoustic image on an ultrasonic image again by a simple operation performed on the probe 102.

Note that, although the display controller 305 changes the superimposed state in step S1211 when the movement speed of the probe 102 is within the predetermined range in the foregoing example, the present invention is not limited to this. For example, the display controller 305 may not superimpose the photoacoustic image on the ultrasonic image even in a case where the movement speed of the probe 102 becomes within the predetermined range, the display controller 305 controls the display unit 104 so that the photoacoustic image is not superposed on an ultrasonic image, and thereafter, the movement speed of the probe 102 becomes within the predetermined range. Note that the probe 102 is moved as below, for example, to display the ultrasonic image on which the photoacoustic image is superimposed. The probe 102 is moved such that the movement speed exceeds an upper limit of the predetermined range, and thereafter, the probe 102 is moved such that the movement speed becomes equal to or lower than the predetermined value used in step S601. Specifically, the display controller 305 displays the ultrasonic image on which the photoacoustic image is superimposed on the display unit 104 when the determination unit 304 determines that the movement speed of the probe 102 becomes equal to or lower than the threshold value used in step S601 after the movement speed of the probe 102 exceeds the upper limit of the predetermined range. Accordingly, display of an ultrasonic image on which a photoacoustic image is not superimposed may remain even when the probe 102 is stopped or moved little in a case where an ultrasonic image on which a photoacoustic image is superimposed is switched to an ultrasonic image on which a photoacoustic image is not superimposed.

Specifically, according to the mode described above, the photoacoustic image may not be easily superimposed on the ultrasonic image after change is made such that the photoacoustic image is not superimposed on the ultrasonic image, and therefore, the user may concentrate on the observation of the ultrasonic image since the user is not disturbed by an operation performed on the probe 102.

Furthermore, although the display controller 305 changes the superimposed state of the photoacoustic image when the movement speed of the probe 102 is higher than the predetermined value and the pressure applied to the probe 102 toward the object is equal to or larger than the predetermined value, the present invention is not limited to this. For example, the display controller 305 may not superimpose the photoacoustic image on the ultrasonic image even in a case where the movement speed of the probe 102 becomes higher than the predetermined value and the pressure applied to the probe 102 toward the object becomes equal to or larger than the predetermined value after the movement speed of the probe 102 is larger than the predetermined value, the pressure applied to the probe 102 toward the object becomes equal to or larger than the predetermined value, and the display controller 305 controls the display unit 104 so that the photoacoustic image is not superimposed on the ultrasonic image. Note that the probe 102 is operated as below, for example, to display the ultrasonic image on which the photoacoustic image is superimposed on the display unit 104. After the pressure applied to the probe 102 toward the object is set smaller than the predetermined value, the movement speed of the probe 102 becomes equal to or lower than the predetermined value and the pressure applied to the probe 102 toward the object becomes equal to or larger than the predetermined value. In this case, the display controller 305 displays the photoacoustic image superposed on the ultrasonic image on the display unit 104 again.

According to the mode described above, the photoacoustic image may not be easily superimposed on the ultrasonic image after change is made such that the photoacoustic image is not superimposed on the ultrasonic image, and therefore, the user may concentrate on the observation of the ultrasonic image since the user is not disturbed by an operation performed on the probe 102.

Furthermore, although the superimposed state of the photoacoustic image is changed when the movement speed of the probe 102 is equal to or lower than the predetermined value and a change of an angle of the probe 102 in a predetermined period of time is equal to or larger than a predetermined value, the present invention is not limited to this. For example, the display controller 305 may not superimpose the photoacoustic image on the ultrasonic image even in a case where the movement speed of the probe 102 becomes equal to or lower than the predetermined value and the change of the angle of the probe 102 in the predetermined period of time becomes equal to or larger than the predetermined value after the movement speed of the probe 102 is equal to or lower than the predetermined value, the change of the angle of the probe 102 for the predetermined period of time is equal to or larger than the predetermined value, and the display controller 305 controls the display unit 104 so that the photoacoustic image is not superimposed on the ultrasonic image. Note that the probe 102 is operated as below, for example, to display the ultrasonic image on which the photoacoustic image is superimposed on the display unit 104 again. For example, the movement speed of the probe 102 is set equal to or lower than the predetermined value after the movement speed of the probe 102 becomes equal to or higher than the predetermined value. Specifically, the display controller 305 displays the ultrasonic image on which the photoacoustic image is superimposed on the display unit 104 when the determination unit 304 determines that the movement speed of the probe 102 becomes equal to or lower than the threshold value after the movement speed of the probe 102 becomes higher than the predetermined value. Accordingly, display of the ultrasonic image on which the photoacoustic image is superimposed may remain even when the angle of the probe 102 is changed in a state in which the probe 102 is not moved or is moved a little.

According to the mode described above, the photoacoustic image may not be easily superimposed on the ultrasonic image after change is made such that the photoacoustic image is not superimposed on the ultrasonic image, and therefore, the user may concentrate on the observation of the ultrasonic image since the user is not disturbed by an operation performed on the probe 102.

The present invention may be realized by a process of supplying a program which realizes at least one of the functions in the foregoing embodiments to a system or an apparatus through a network or a storage medium and reading and executing the program using at least one processor of a computer included in the system or the apparatus. Furthermore, the present invention may be realized by a circuit which realizes at least one of the functions (an application specific integrated circuit (ASIC), for example).

The control device in each of the foregoing embodiments may be realized as a single device or a plurality of devices are combined with each other in a communication available manner so as to realize the process described above. Both the cases are included in embodiments of the present invention. Alternatively, the process described above may be executed by a common server apparatus or a server group. The control device and the plurality of devices included in the control system may at least communicate with each other at a predetermined communication rate and may not be included in the same facility or the same country.

Embodiments of the present invention include a mode in which a software program which realizes the functions of the foregoing embodiments is supplied to a system or an apparatus and a computer included in the system or the apparatus reads and executes a code of the supplied program.

Accordingly, the program code installed in the computer so as to realize processes according to the embodiments using the computer is also an embodiment of the present invention. Furthermore, an operating system (OS) which operates in the computer actually performs a portion of or the entire process based on an instruction included in a program read by the computer and the functions of the foregoing embodiments may be realized by the process.

A modes obtained by appropriately combining the foregoing embodiments is also an embodiment of the present invention.

According to the present invention, a photoacoustic image generated using a photoacoustic signal based on information on a movement of a probe may be displayed on a display unit, and therefore, an operation of performing switching of an operation mode associated with detection of an ultrasonic signal and the photoacoustic signal may be omitted.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. A control device, comprising:
first obtaining means for obtaining an ultrasonic signal and a photoacoustic signal using a probe which outputs the ultrasonic signal by transmission and reception of an ultrasonic wave relative to a test object and which outputs the photoacoustic signal by receiving a photoacoustic wave generated due to light irradiation onto the test object;
second obtaining means for obtaining information on displacement of the probe;
determination means for determining whether the photoacoustic image is to be displayed in a display unit based on the information on displacement of the probe; and
display control means for displaying a photoacoustic image generated using the photoacoustic signal based on a result of the determination indicating that the photoacoustic image is to be displayed in the display unit performed by the determination unit.

2. The control device according to claim 1, wherein the display control means displays the photoacoustic image based on the information on displacement when an ultrasonic image generated using the ultrasonic signal is being displayed in the display unit.

3. The control device according to claim 1, wherein the second obtaining means obtains, as the information on displacement, at least one of information on a position and an orientation of the probe relative to the test object, information on a movement speed of the probe relative to the test object, information on a rotation speed of the probe, information on acceleration of a movement relative to the test object, and information indicating a degree of pressure relative to the test object.

4. The control device according to claim 1, wherein the display control means displays the photoacoustic image in the display unit when at least one of information indicating that the probe is moved at a speed lower than a predetermined speed relative to the test object and information indicating that the probe is pressed to the test object at a constant pressure is obtained.

5. The control device according to claim 1, wherein the display control means differentiates a mode of the photoacoustic image displayed in the display unit in accordance with a degree of the displacement.

6. The control device according to claim 5, wherein the display control means displays the photoacoustic image in the display unit such that transparency of the photoacoustic image is increased as a movement speed relative to the test object is higher.

7. The control device according to claim 1, wherein the determination means determines that the photoacoustic image is to be displayed in the display unit when the obtaining means obtains at least one of information indicating that the probe is moved at a speed lower than a predetermined speed relative to the test object and information indicating that the probe is pressed to the test object in a pressure higher than a predetermined pressure.

8. The control device according to claim 1, further comprising irradiation control means for controlling an irradiation unit so that the irradiation unit irradiates the test object with light when the determination unit determines that the photoacoustic image is to be displayed in the display unit.

9. The control device according to claim 1, further comprising generation means for generating an ultrasonic image based on the ultrasonic signal obtained by the first obtaining means and generates a photoacoustic image based on the photoacoustic signal.

10. The control device according to claim 9 further comprising output means for outputting the ultrasonic image and the photoacoustic image which are generated by the generation means and which are associated with each other to an external apparatus.

11. The control device according to claim 10, wherein the output means outputs information for associating the ultrasonic image with the photoacoustic image which is attached to the ultrasonic image and the photoacoustic image.

12. The control device according to claim 9 further comprising output means for outputting a superimposed image obtained by superimposing the photoacoustic image on the ultrasonic image generated by the generation means to an external apparatus.

13. The control device according to claim 10 wherein the output means attaches information indicating apposition of the probe which has obtained the ultrasonic signal for generating the ultrasonic image to the ultrasonic image.

14. The control device according to claim 10, wherein the output means attaches the information indicating a position of the probe which has obtained the photoacoustic signal for generating the photoacoustic image to the photoacoustic image.

15. The control device according to claim 1, further comprising guide means for generating guide information for guiding the probe to a specific position.

16. The control device according to claim 1, further comprising notification means for making a notification indicating that the probe performs the light irradiation to obtain the photoacoustic signal.

17. The control device according to claim 1, wherein the display control means displays an ultrasonic image generated from the ultrasonic signal in the display unit and displays the photoacoustic image superimposed on the ultrasonic image based on the information on displacement of the probe.

18. The control device according to claim 1, wherein the second obtaining means obtains information on displacement of the probe in a magnetic field based on information obtained from a magnetic sensor included in the probe.

19. The control device according to claim 1, wherein the second obtaining means obtains information on displacement of the probe based on information obtained by a pressure sensor included in the probe.

20. The control device according to claim 1, wherein the information on displacement of the probe corresponds to a mode for a user to operate the probe.

21. A control method, comprising:
a step of obtaining information on displacement of a probe which outputs an ultrasonic signal by transmission and reception of an ultrasonic wave relative to a test object and which outputs a photoacoustic signal by receiving a photoacoustic wave generated due to light irradiation onto the test object;
a step of determining whether the photoacoustic image generated using the photoacoustic signal is to be displayed in a display unit based on the information on displacement of the probe; and
a step of displaying the photoacoustic image in the display unit based on a result of the determination indicating that the photoacoustic image is to be displayed in the display unit performed by the determination unit.

22. A non-transitory storage medium that stores a program that causes a computer to execute the control method according to claim 21.
